(19) United States
(12) Patent Application Publication
(10) Pub. No.: US 2007/0173892 A1
(43) Pub. Date: Jul. 26, 2007

(54) Method and an apparatus for measuring of physiological parameters

(76) Inventors: Jesper Fleischer, Hojbjerg (DK); Martin S. Jensen, Aarhus C. (DK)

Correspondence Address: DYKEMA GOSSETT PLLC, FRANKLIN SQUARE, THIRD FLOOR WEST, 1300 I STREET, NW, WASHINGTON, DC, 20005, US

(21) Appl. No.: 11/516,663

(22) Filed: Sep. 7, 2006

Related U.S. Application Data
(63) Continuation-in-part of application No. PCT/DK05/00102, filed on Feb. 17, 2005.

(30) Foreign Application Priority Data
Mar. 8, 2004 (DK) PA 2004 00385

(51) Int. Cl. A61B 5/04; A61N 1/00; A61B 5/02; A61B 5/05

(52) U.S. Cl. 607/2; 600/547; 600/500; 600/393

(57) ABSTRACT

The invention relates to an active plaster capable of applying electric current with a view to promoting a wound healing process in humans or animals based on the use of electrodes and electronics incorporated in a plaster which encapsulates the wound. It is moreover contemplated by the invention that the active plaster may contain electronics for measuring and indicating the wound stage. One or more diodes on the active plaster and/or on an external apparatus, which may be connected to the plaster, emit a green, yellow or red light and indicate which stage the wound is in, and thereby whether the plaster is to be changed. The measurements are based on the use of an apparatus with firmly integrated electrodes and electronics capable of applying current in the measurement object, such as for detecting the uptake of liquid in the tissue, determining the degree of ischemia and thereby determining the wound healing stage. The apparatus forming part of the invention is incorporated in a plaster which is adhered directly on top of the surface wound by a self-adhesive material, and which encapsulates the wound.

The invention relates to a method of measuring physiological parameters on humans or animals based on the application of electrodes including electrodes for contact with the skin surface.

The invention moreover relates to a method of measuring physiological parameters in humans or animals based on the use of electrodes, including electrodes for contact with the surface of the skin,

The invention moreover relates to an apparatus for measuring of physiological parameters on humans or animals based on the application of electrodes including electrodes for contact with the skin surface.

Finally the invention relates to a device for measuring physiological parameters in humans or animals based on the use of electrodes, including electrodes for contact with the surface of the skin

It is known to measure physiological parameters by using electrodes including the use of measuring impedance. The known technique includes the use of electrodes, which are attached to the skin surface around the measuring object, where the electrodes afterwards are connected with wires to the actual measuring apparatus.

The background for using impedance measurements is that all tissue and all organs have a characteristic impedance, measurement of impedance in a given object being measured can therefore give information about the composition of the tissue in the object being measured. At the same time the measurement is sensitive towards physiological changes in the object being measured, which means, that measurement of impedance often, with advantage, can be used in detecting such changes.

One of the major advantages of using measurements of impedance based on electrodes, which are attached to the surface of the skin, is that the measurements are non-invasive and are only making the people or animals being measured feel discomfort to a very small extent.

It has been found, however, that there are some drawbacks associated with the known technique.

The procedure with application of loose electrodes, which are being attached to the skin surface and after that being connected with wires to the actual measuring apparatus gives as an example the drawback that the measurements are hard to reproduce given that the mutual location of the electrodes only with much difficulty can be recreated with high precision.

Due to the use of loose electrodes the geometry between the electrodes will vary from measurement to measurement, which means, that the uncertainty of the individual measurement is relatively high.

The measurement is in addition complicated due to the use of wires which, besides being awkward to handle, easily can break or give periodic poor contact and thus give a cause for erroneous measurement.

It is as well a drawback that the equipment due to the use of loose electrodes, which are attached to the measuring apparatus with wires, becomes unmanageable and troublesome to transport, as the whole of the measuring apparatus consists of many separate parts.

Accordingly, an object of the invention is to improve the known procedure and the known apparatus.

The object of the invention is achieved by a method of the in the introduction to claim 1 stated type, which is characteristic by that the electrodes are mutually retained in a fixed geometry.

Hereby, it is thus possible to get a high precision on every single measurement, in that the geometry of measuring always is known and always is the same, as well as it gets easier to reproduce measurements.

In claim 2 is stated that it is as well a distinctive feature of the invention that the electrodes are integrated in a measuring device, which can be hand-held or fastened to the point of measurement such as by using a band or an adhesive substance.

When the electrodes are integrated in the measuring apparatus, the wires, which are normally used for connecting the electrodes with the measuring apparatus, are removed, and thus the number of potential errors decrease by which the quality of measurement is increased, as well as the equipment is easily operated hand-held. If the whole apparatus is attached to the object being measured, a person can as an example be monitored continuous in one's everyday life, which opens op for entirely new possibilities of measuring- and application methods.

As stated in claim 3 it is moreover a distinctive feature of the invention that the electrodes are used for measurement of impedance, where one or more, preferably two, are used for signal-generation, and where one or more, preferably two, are used for signal detection.

Hereby it is achieved that the measurement of impedance can be executed with high precision and a high extent of reproducibility, which totally gives measured data with relatively low uncertainty and thus a high extent of practical applicability.

Further preferred embodiments of the method of the invention are defined in claims 4 to 8.

As mentioned the invention also relates to an apparatus.

This apparatus or instrument is characteristic in that it can be operated hand-held or attached to the point of measurement with a band such as a so called velcro band or with an adhesive substance, and is supplied with two or more integrated electrodes for contact with the skin surface and can be programmable and can be provided with indicators or a display for visualization of the measured physiological parameters and means for storage of data and data communication with external digital units such as computers.

Hereby it becomes possible, simple and effective, to measure physiological parameters, including impedance based, with high precision and reproducibility, which results in large practical, including clinical, applicability. Simultaneously it becomes possible to long-term monitor people or animals in their usual surrounding environment.

Further expedient embodiments are defined in claim 6-10.

As mentioned the invention relates to another method and a device. This method is characterized in that the electrodes and the electronics are integrated in the measuring equipment, which is adhered on top of a surface wound using an adhesive, and the device is characterized in that it may be adhered on top of a surface wound by an adhesive and is provided with two or more integrated electrodes for contact with the surface of the skin and may be programmable and may be provided with indicators or a display for visualizing the measured physiological parameters and means for data storage and data communication with external units, such as computers.

Embodiments for this method are defined in claims 12-14, whereas embodiment for the apparatus is defined in claim 16-18.

In the following the prior art relating to claims 11-18 can be summarized as follows:

The invention relates to an active plaster which is capable of applying electric current with a view to promoting a wound healing process in humans or animals based on the use of electrodes and electronics incorporated in a plaster which encapsulates the wound.

It is moreover contemplated by the invention that the active plaster may contain electronics for measuring and indicating the wound stage. One or more diodes on the active plaster give out e.g. a green, yellow or red light and indicate thereby in which stage the wound is, and thereby whether the plaster is to be changed.

The risk of getting a problem wound—a wound that will not heal—increases with the age and with a poor blood circulation. The most common type of problem wounds is ankle ulcers which are most frequently located around the malleolus. The cause of these problem wounds is the upright position of the individual, which means that the blood column from leg to heart causes an elevated blood pressure in the veins of the leg. With age, this pressure may become a too great load on the valves of the veins of the leg, which therefore cannot close tightly. In case of poorly functioning venous valves, the blood flows toward locations having the smallest pressure, i.e. to the surface veins of the legs. They are dilated hereby, and varices are formed. Other types of wounds are e.g. leg sores and pressure sores (bed sores)

Diabetics and individuals having a too high blood pressure and a too high fat content in the blood are particularly susceptible to leg sores, including foot sores, and the risk is enhanced if the individual smokes. Diabetics in particular have an enhanced risk of foot sores, which may be a symptom of a concealed late complication, such as peripheral neuropathy. Other causes are e.g. ischemia because of arteriosclerosis and reduced infection control capacity.

Particularly susceptible to pressure sores are the elderly, individuals suffering from paralysis, heart diseases, diabetes, and individuals who are unconscious, emaciated or overweight. Liquid deficiency can enhance the risk.

In addition to life quality loss of the individual because of chronic wounds, patients having problem wounds are expensive to the society, because today the treatment is complicated and time-consuming. Today, wound treatment accounts for 20-30 percent of the work of the visiting nurses in Denmark. In particular many elderly individuals sustain chronic wounds. Since life expectancy and particularly the number of diabetics increase by up to 20% per year, the problem and the costs will increase in future.

It is known to measure physiological parameters using electrodes, including using impedance measurements. It is likewise known to apply current to promote a wound healing process.

Research has shown that treatments where a very weak direct current or alternating current is passed through a wound, has a healing effect. The literature thus includes many articles on the beneficial effect of electric current inter alia by increasing the metabolism.

Another described beneficial effect of the treatment with electric current is the reduction in the concentration of bacteria promoting ions. The cells in elderly people contain a greater concentration of Mg and Zn ions. These ions have a great impact on the formation of bacteria and can perhaps explain the poorer wound healing and enhanced risk of infections which are seen in elderly people. The electrolysis caused by the current treatment reduces the concentration of these ions.

The prior art concerning wound healing (US 2005/0119715; FR 2849772; WO 2006045054; US 2003130707; U.S. Pat. No. 5,861,016; WO 9701372; U.S. Pat. No. 5,230,703; U.S. Pat. No. 5,158,081; US 469484) and/or bioimpedance measurement (WO 0028892; U.S. Pat. No. 3,870,034; U.S. Pat. No. 4,949,727; GB 1556749; EP 1219238) comprises the use of electrodes which are adhered to the surface of the skin around a surface wound and/or the measurement object, following which the electrodes are connected by wires to the actual measuring apparatus.

The patent US 2005/0119715 describes a wound healing system, which is characterized in that the external electrodes are capable of applying current as well as measuring the impedance in the wound. In addition, the importance of locating the electrodes so as to make the current run through the wound, is described in detail. The impedance measurement is used in the apparatus for adjusting the electrical resistance and thereby the current which is passed through the wound. The patent also states that the impedance may be measured during several treatments, be saved and used for calculating the wound healing rate. The patent also mentions that the apparatus must be capable of being used in the patient's own home.

However, it has been found that the prior art is vitiated by some drawbacks.

The method of using loose electrodes which are applied to the surface of the skin around the wound, e.g. involves the drawback that the wound is not encapsulated and will thereby lose moisture.

Modern wound treatment is based on the principle of moist wound healing. This is in direct contrast to the previous dry wound treatment. One of the decisive factors of the more recent wound treatment is that the frequency at which the plaster/dressing is changed, is reduced as much as possible. Encapsulation of the wound increases the epithelialization rate by a factor 2-3. Therefore, it is of paramount importance to the invention that the wound is covered by a plaster.

However, in the inflammation phase, in particular, it is important to change the dressing if the wound runs. The healing process depends on the moisture of the wound, the balance point being to change the dressing of the running wound before the environment favours pathogenic microorganisms.

An indicator, such as e.g. a diode on the active plaster which gives out a green, yellow or red light, indicates to which degree the wound runs, and thereby whether the dressing is to be changed. This important indication option is to ensure that the plaster is not changed unnecessarily. Indication on the tissue state may also be measured by means of an external apparatus which is connected to the plaster. This option is particularly intended for professional use.

To reduce the consumption of power as much as possible, the active plaster is provided with a switch/connector which is to be short-circuited before the diode emits light. This connector may also be used for connecting other measuring equipment for the active plaster.

It is also a great drawback that the equipment consists of several separate parts, including loose electrodes which are connected to the measuring apparatus by wires. This makes the equipment unhandy and is not conducive to making the patient apply this very beneficial treatment form in his normal everyday life.

The invention will now be explained more fully with reference to the drawings, in which:

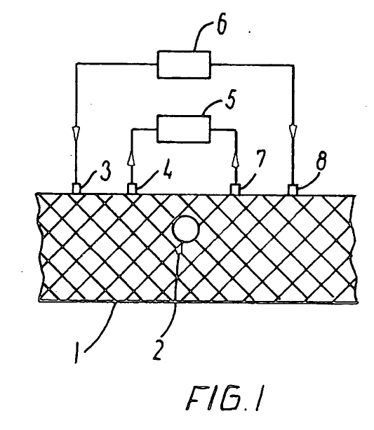

FIG. 1 shows an elementary sketch of a measurement of impedance for determination of physiological parameters.

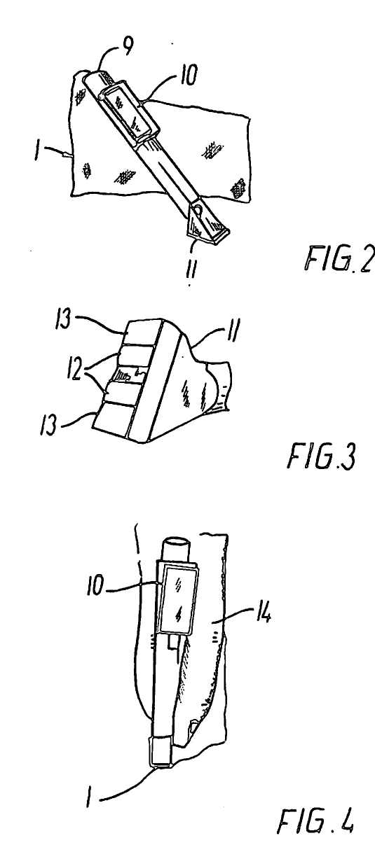

FIG. 2 shows a hand-held apparatus for measurement of physiological parameters on an object under measurement in the form of a forearm.

FIG. 3 shows the transducerhead from the measurement equipment shown in FIG. 1.

FIG. 4 shows a practical measuring setup where a hand-held measurement device is used for measuring physiological parameters on by way of example the forearm of a human.

FIG. 5 shows, seen from the side, a hand-held measuring apparatus supplied with integrated electrodes in the bottom and control buttons and display in the top.

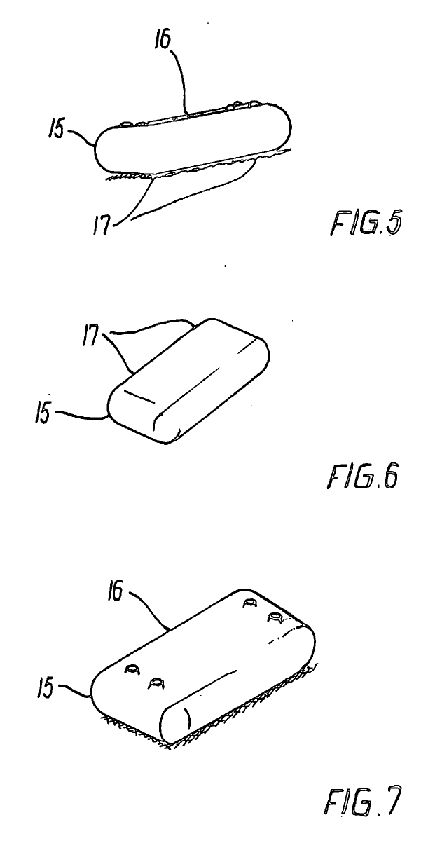

FIG. 6 shows, seen from the bottom, the same measurement device as FIG. 5.

FIG. 7 shows, seen from above, the same measurement device as FIG. 5 and FIG. 6.

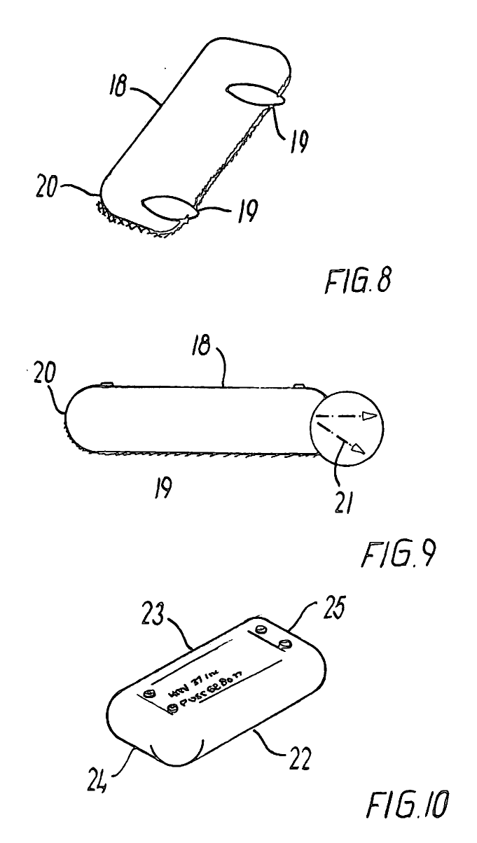

FIG. 8 shows, seen from the bottom, a hand-held measurement device with numerous rows of integrated electrodes, which can be used for impedance tomography.

FIG. 9 shows, seen from the side, the same measurement device as FIG. 8.

FIG. 10 shows a measurement device with electrodes integrated in the lateral surfaces of the apparatus.

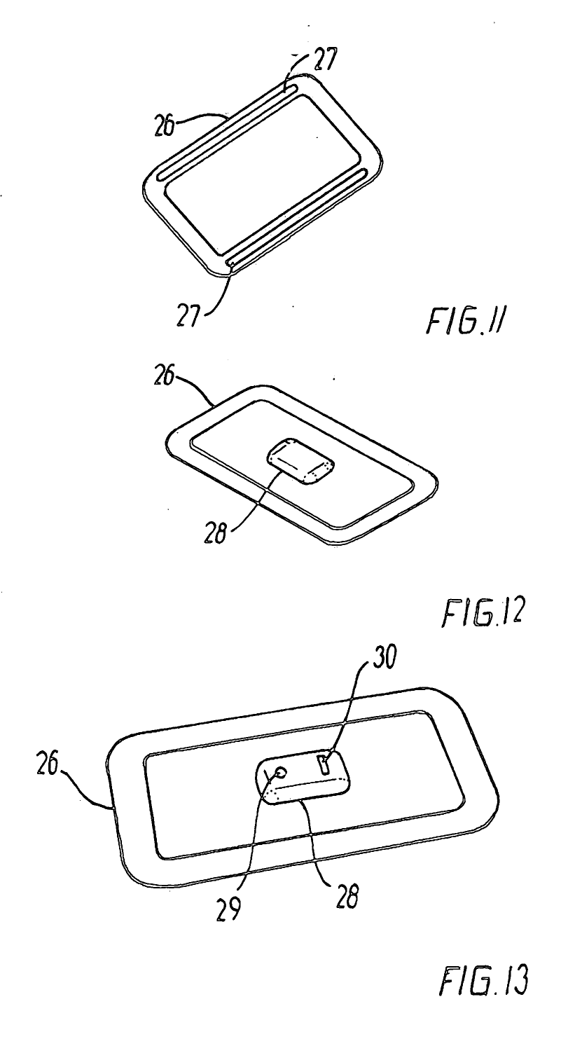

FIG. 11 shows, seen from the bottom, an apparatus with two integrated electrodes for fixing to a surface of skin.

FIG. 12 shows, seen from the top, the same apparatus as shown on FIG. 11 and

FIG. 13 shows an embodiment of a basic sketch of an active plaster, according to the invention.

In FIG. 1, 1 indicates a cross section of a measurement device, e.g. in form of a segment of a humane forearm, which is penetrated by an artery 2.

On the surface of the object of measurement, which, in the example, is the surface of the persons skin, electrodes are placed 3, 4, 7 and 8.

The electrodes 4 and 7 are connected to a signal detection circuit 5, while electrodes 3 and 8 are connected to a signal generation circuit 6.

In practice the signal generation circuit 6 is a source of electrical current, where alternating current is preferred, while the signal detection circuit is a voltage detector.

During a measurement, the signal, being the alternating current, which from 6 is penetrating the measurement object 1, as well as the resulting voltage, which is registered in the detection circuit 5 is known. With knowledge of the current through, as well as the voltage across the object of measurement the impedance can simply be derived based on the formula R=V/I, where:

R is the impedance in the volume of measurement, which is covered by the measurement setup,

V is the voltage, which is measured in 5 and

I is the current, which is generated from 6.

The cubical content, which is defined by the measuring set-up, is primarily dependant of the mutual location of the electrodes and their physical extent.

As a result of the person's heartbeat the blood will be pumped through the blood vessels of the body and thus result in pulsation of the artery 2, whereby the shown cross-sectional area will change as a function of the heartbeats.

Due to the fact that the blood, which flows through the artery, has got characteristic impedance than the nearby tissue, the artery pulsation can be registered in the signal detector 5.

By using a simple signal algorithm, the heart rate can thereby relatively easily be derived from the signal detector 5. From the heart rate signal additional parameters such as HRV (Heart Rate Variability), including short-time measurement of HRV (short-term HRV) can as well be derived.

In FIG. 2 an apparatus consisting of a measuring probe 9 is shown, which can be one-hand operated and thus it can be defined as hand-held, which is supplied with a display 10 for presentation of the measured and/or derived physiological parameters such as the pulse rate of the heart or HRV. The measuring probe 9 is supplied with a probehead 11, which contains electrodes for generation of signals and signal detection, whereby impedance based physiological parameters can be derived from an object of measurement, e.g. by way of a humane forearm 1.

FIG. 3 shows, seen from the bottom and in an enlarged illustration, the probehead 11 from the measuring probe 10 shown in FIG. 2.

The probehead is provided with two electrodes 13, which are being used for emitting current in the object of measurement, and two electrodes 12, which are being used to detect the voltage, which is created as a function of the current in the object of measurement.

In advance of a measurement, the probehead 11 is placed on the skin over the area of measurement, such that the electrodes 12 and 13 are in direct contact with the surface of the skin.

In FIG. 4 an example is shown of a practical measurement of physiological parameters such as heart rate and HRV from the forearm of a humane object under measurement 1, by application of a measurement probe 10, which is operated with a hand 14 by the user.

The registered and/or derived physiological parameters are shown on the display of the probe 10.

Another example of a preferred embodiment of the present invention is shown in FIG. 5. The measurement device 15 can be operated with one hand and is at the top provided with operating buttons and a display for data visualization, and at the bottom provided with integrated electrodes 17. The device 15 is shown seen from the bottom in FIG. 6 and seen from above in FIG. 7.

As it will appear from FIGS. 5 to 7, the device 15 is characterized by, that the electrodes 17 are integrated in the product and therefore mutually placed in a fixed geometry. By this the geometry of measurement is always well-defined and the measurement results can therefore be defined and reproduced with high precision. The reproducibility is of crucial significance in many applications, as an example in case the measured or derived physiological parameters are going to be used in clinical applications.

It is in addition seen that the device 15 is easy to operate, easy to clean and easy to transport. Because the electrodes 17 are integrated in the product there are no wires, which must be connected between the electrodes and the device, and thus the quantity of potential sources of error are reduced significantly.

Apparatus, as the shown example 15, can, depending of the number of electrodes 17 and the mutual geometrical position of these, be optimized for measuring of different types of physiological parameters derived in a well-defined including limited volume such as determination of mass of fatty tissue, determination of degree of ischaemia, determination of absorption of liquid and fluid in tissue and determination of stage or degree of wound healing.

In case there is being measured across an artery as shown schematic in FIG. 1 in form of an artery, there can, as previously mentioned, be derived data about heart rate and HRV, as well as it is possible to derive information about the blood flow in the vein and the respiration of the person on basis of the measured signals.

In FIG. 8 an apparatus 20 is shown, seen from the bottom, which is provided with an array 19 of electrodes. By using an array of electrodes it is possible to get a three-dimensional tomographic identification of the tissue defined by the measurement apparatus 20.

In a preferred embodiment of an impedance tomographic measurement device 20 the marked outer rows of electrodes 19 are the source of current, while the intermediate electrodes are used for signal detection.

With a construction as the one shown in FIG. 8 of a device 20 it is possible to detect form, size and location of an underlying vein or artery. In FIG. 9 such an impedance tomographic device 20 is shown, which is provided with one or more sources of light 21, which can be used to mark, with a spot of light on the surface of the skin, e.g. the centre of an underlying artery. Such a mark can later be used as a target point for inserting a hypodermic needle into the artery.

In FIG. 10 another preferred embodiment of the present invention is shown consisting of a device 24, which is provided with two integrated electrodes 24 and 25, which can be used for registering the self induced or natural electrical voltage of a object of measurement, as an example in form of a human, generates in response to the electrocardiographical (ECG) signal, which controls the contraction of the heart.

Measurement of impedance performed with the same electrodes, which record the ECG signal, can be used to determine if the device is in touch with an individual, which e.g. can be used to determine the beginning as well as the ending of an ECG measurement.

If a person with each hand holds on to respectively electrode 24 and electrode 25, the ECG signal can easily be detected, whereby physiological parameters such as heart rate and HRV including short-time HRV can easily be derived and presented on the built-in display 23.

FIG. 11 shows an example of a device 26 carried out in pursuant to the invention. The device is seen from the bottom where the device is provided with two integrated electrodes 27. The device 26 is in FIG. 12 shown from above, where it is provided with a unit 28, which can typically contain electronics, battery and e.g. light-emitting diodes.

As it will appear from FIG. 11 and FIG. 12 the device is thin in relation to the length and width. In practice the device can be made of a flexible thin material and can be applied with a self-adhesive substance, thus the whole device 26 can be attached to the skin surface of a person.

If the device is placed above a surface wound, the electrodes 27 can be used partially for emitting current through the wound, which can often have a positive effect on the wound healing, as well as the electrodes can be shaped so they can also measure the impedance in the area of the wound.

The impedance in the area of the wound will be a function of the wound healing stage, which thus can be shown on 28, e.g. by one or more indicators such as light-emitting diodes. If light-emitting diodes are used the colour of these could e.g. change, in such a way that an open wound would give a red light, while a healing wound would be shown in yellow towards green colours dependant of the stage of the wound healing.

FIG. 13 shows a basic sketch of the active plaster 26 embodied according to the invention. The apparatus is shown from above, it being provided with a unit 28 which contains a battery, a light emitting diode 29 and control electronics, including a microprocessor or microcontroller, communications circuits so that data may be exchanged, e.g. wirelessly, with external units, such as computers. The switch/connector 30 is used as a current-saving feature for the diodes and as an input to external measuring equipment.

As will appear from FIG. 11, FIG. 12 and FIG. 13, the apparatus is thin relative to the length and width. In practice, the apparatus may be made of a flexible thin material and have a self-adhesive substance applied thereon, so that the entire apparatus 26 may be carried without problems like a normal plaster in the patient's everyday life.

The apparatus is adhered directly on top of the surface wound by a self-adhesive material and encapsulates the wound and thereby retains the important moisture. The electrodes 27 are used partly for passing a current through the wound, which has a beneficial effect on the wound healing.

Indication of the wound healing stage is shown by means of one or more light emitting diodes 29 and is turned on by short-circuiting the switch 30. The colour of these diodes may e.g. change, so that the running wound emits a red light, while wound healing is shown in yellow toward green colours depending on the wound healing stage.

Thus, it is part of the present invention that the electrode-based apparatus may be used both for measuring physiological parameters, but also for applying current with a view to promoting a wound healing process.

The apparatus is sterilized and is made of materials which are not harmful to the skin and the environment.

The invention is not restricted to the embodiments which are directly described and/or shown in the figures, but also encompasses all embodiments which may be derived indirectly from the present text or the present figures.

It is thus a part of the present invention that the electrode based device can be used for measuring of physiological parameters as well as for emitting current with regard to e.g. advance wound healing processes or to stimulate muscles.

It is also a part of the present invention that the device is hand-held or can be attached to the object of measurement as an example by using band, such as Velcro, tape or self-adhensive material.

All the devices can be sterilized and manufactured by skin- and environmentally friendly materials.

All the shown examples of devices can be programmable, typically by use of a microprocessor or—controller, and all the devices can be provided with communication circuits, so that data can be exchanged e.g. wireless with external units such as computers.

The invention is not limited to the forms of construction, which are directly described and/or shown in the figures, but also covers all forms of construction, which can indirectly be derived from the present text or the present figures.

1. A method of measuring physiological parameters on humans or animals based on the application of electrodes including electrodes for contact with the surface of the skin wherein the electrodes are mutually retained in a fixed geometry.

2. A method according to claim 1, wherein the electrodes are integrated in a measurement device, which can be hand-held or attached to the place of measurement such as by using a band or a self-adhesive material.

3. A method according to claim 1 wherein the electrodes are used for measurement of impedance, where one or more including preferably two are used to generate signals and where one or more including preferably two are used for signal detection.

4. A method according to claim 1, wherein heart rate and/or HRV including short-time HRV are measured.

5. A method according to claim 1, wherein the physiological parameters which are being measured can be determined in a well-defined including limited volume and comprise parameters such as thickness of tissue including fatty tissue, condition of tissue including the content of liquid such as water, degree of ischaemia, wound healing stages or flow of liquid including blood flow in veins such as arteries.

6. A method according to claim 1, wherein the measurement devices can be programmable and can be provided with indicators or a display for visualization of the measured physiological parameters and means for storage of data and data communication with external digital units such as computers.

7. A method according to claim 1, wherein the veins and arteries can be detected and marked on the surface of the skin for instance by using light.

8. A method according to claim 1, wherein two or more of the electrodes can be used for emitting electrical energy to stimulate tissue such as stimuli of muscles or healing wounds.

9. An apparatus for measuring physiological parameters on humans or animals based on the application of electrodes including electrodes for contact with the surface of the skin wherein it can be operated hand-held or attached to the point of measurement with a band such as a velcro band or with an adhesive substance, and is supplied with two or more integrated electrodes for contact with the skin surface and can be programmable and can be provided with indicators or a display for visualization of the measured physiological parameters and means for storage of data and data communication with external digital units such as computers.

10. An apparatus according to claim 9, wherein the product contains two or more electrodes, which can emit electrical energy to stimulate tissue including stimuli of muscles or wound healing.

11. A method of measuring physiological parameters in humans or animals based on the use of electrodes, including electrodes for contact with the surface of the skin, wherein the electrodes and the electronics are integrated in the measuring equipment, which is adhered on top of a surface wound using an adhesive.

12. A method according to claim 11, wherein the physiological parameters, which are measured, are determined in a well-defined, including limited volume, and comprise parameters, such as the tissue state, including the content of liquid, the degree of ischemia and thereby the wound healing stage.

13. A method according to claim 11, wherein the measuring equipment may be programmable and may be provided with indicators or a display for visualizing the measured physiological parameters, and means for data storage and data communication with external digital units, such as computers.

14. A method according to claim 11, wherein two or more of the electrodes may be used for applying electrical energy for wound healing.

15. A device for measuring physiological parameters in humans or animals based on the use of electrodes, including electrodes for contact with the surface of the skin, wherein the device may be adhered on top of a surface wound by an adhesive and is provided with two or more integrated electrodes for contact with the surface of the skin and may be programmable and may be provided with indicators or a display for visualizing the measured physiological parameters and means for data storage and data communication with external units, such as computers.

16. A device according to claim 15, wherein it consists of a plaster.

17. A device according to claim 15, wherein the indicators displays a wound healing stage.

18. A device according to claim 15, wherein the device contains two or more electrodes which are capable of applying electrical energy for wound healing.
